(19) United States
(12) Patent Application Publication
(10) Pub. No.: US 2021/0386291 A1
(43) Pub. Date: Dec. 16, 2021

(54) WEARABLE DEVICE WITH CLOUD-BASED MONITORING SOFTWARE

(71) Applicant: AIRES MEDICAL LLC, Ann Arbor, MI (US)

(72) Inventors: Nicholas Leonard Oddo, Hilton Head Island, SC (US); Shane Woody, Mooresville, NC (US); Chad Josey, Mooresville, NC (US); Dylan Moore, Winter Park, FL (US)

(73) Assignee: AIRES MEDICAL LLC, Ann Arbor, MI (US)

(21) Appl. No.: 17/458,740

(22) Filed: Aug. 27, 2021

Related U.S. Application Data
(63) Continuation-in-part of application No. 16/996,063, filed on Aug. 18, 2020, now Pat. No. 11,123,505.
Application No. 16/996,063 is a continuation-in-part of application No. 16/704,413, filed on Dec. 5, 2019, now Pat. No. 10,946,161.
(60) Provisional application No. 63/047,742, filed on Jul. 2, 2020.
(60) Provisional application No. 62/775,733, filed on Dec. 5, 2018.

(51) Int. Cl. A61B 5/00; A61M 16/00; A63B 24/00; G16H 40/67; G16H 20/13

(52) U.S. Cl. CPC A61B 5/0022; A61M 16/024; A61B 5/4839; A61B 5/681; A61B 5/6826; G16H 20/13; A61B 5/742; A61B 5/4806; A61B 5/4857; A63B 24/0062; G16H 40/67; A61B 5/746

(57) ABSTRACT

In an aspect, a system for a cloud-based user physiology detection software and patient monitoring system. A system includes a wearable device. A wearable device includes a sensor. A sensor is configured to receive physiological data from a user. A system includes a patient monitor configured to monitor a vital sign of a user. A system includes a therapeutic delivery device configured to administer a therapeutic remedy to a user. A system includes a computing device configured to modify a therapeutic remedy of a therapeutic delivery device as a function of physiological data. A method of providing a therapeutic remedy using a cloud-based detection software is also disclosed.

### CROSS-REFERENCE TO RELATED APPLICATIONS

This continuation-in-part application claims priority, and the benefit of, U.S. patent application Ser. No. 16/996,063, filed Aug. 18, 2020, which priority, and the benefit of, U.S. Provisional Patent Application 63/047,742, filed Jul. 2, 2020, and U.S. patent application Ser. No. 16/704,413, filed on Dec. 5, 2019, which claims priority, and the benefit of, U.S. Provisional Patent Application 62/775,733, filed on Dec. 5, 2018, each of which is hereby incorporated by reference in its entirety.

### TECHNICAL FIELD

The present disclosure generally relates to a medical device, and more particularly, to a wearable device with a cloud-based network monitoring software.

### BACKGROUND

Some breathing apparatus can lack portability and require continuous monitoring of user condition and manual adjustment of breathing settings by health care personnel. In many cases, expensive breathing monitoring technologies such as CO2 capnography must be used in conjunction with a breathing apparatus, to determine effectiveness and make adjustments in settings during use. Some control methodologies and configurations are not readily adaptable for use with certain user conditions, for example, when the user is talking, during sleep, or when the user is connected to Continuous Positive Airway Pressure (CPAP) and/or Bilevel Positive Airway Pressure (BiPAP) machines, for example, during sleep apnea therapy.

### SUMMARY

In an aspect, a system for a cloud-based user physiology detection software and patient monitoring system. A system includes a wearable device. A wearable device includes a sensor. A sensor is configured to receive physiological data from a user. A system includes a patient monitor configured to monitor a vital sign of a user. A system includes a therapeutic delivery device configured to administer a therapeutic remedy to a user. A system includes a computing device configured to modify a therapeutic remedy of a therapeutic delivery device as a function of physiological data.

In an aspect, a method of providing a therapeutic remedy using a cloud-based detection software. A method includes measuring on a sensor of a wearable device a physiological data of a user. A method includes transmitting to a cloud-based network data of physiological data of a user. A method includes adjusting a therapeutic remedy of a user as a function of physiological data.

### BRIEF DESCRIPTION OF THE DRAWINGS

The accompanying drawings, which are incorporated into and constitute a part of this specification, illustrate implementations of the disclosure and together with the description, serve to explain the principles of the disclosure.

FIG. 1 is a schematic diagram of a breathing apparatus, such as a ventilator.

FIG. 2 is a schematic diagram of an invasive circuit using a breathing tube, an adapter, and an oxygen tubing.

FIG. 3 is a schematic diagram of a breathing apparatus that can function as BiPAP, CPAP device, an O₂ concentration, and/or ventilator with different modes.

FIG. 4 is a breath detection predictive curve software graph.

FIG. 5 is a graph of pressure reading v. time used to identify the breathing state of a user of a breathing apparatus.

FIG. 6 is a tidal volume graph compared with a breathing cycle and a breathing apparatus operation.

FIG. 7 is a schematic diagram of a breathing apparatus with cloud-based breath detection software and patient monitoring.

FIG. 8 is a flowchart of a method for dynamically adjusting tidal volumes of a breathing apparatus based on the PaO₂ saturations and vital sign monitor data.

FIG. 9 is an exemplary embodiment of a block diagram illustrating a wearable device.

FIG. 10 is an exemplary diagram of a machine learning model.

FIG. 11 is a block diagram of a method of monitoring a physiology of a user.

FIG. 12 is an exemplary embodiment of a computing device that may be implemented in any of the methodologies described herein.

### DETAILED DESCRIPTION

The foregoing summary, as well as the following detailed description of certain embodiments will be better understood when read in conjunction with the appended drawings. As used herein, an element or step recited in the singular and preceded by the word “a” or “an” should be understood as not necessarily excluding the plural of the elements or steps. Further, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property. An “electronic communication” as used in this disclosure is any connection through which data signals are transferred between two or more components. A “fluid communication” as used in this disclosure is a pathway between two components that allows a flow of gas and/or fluids

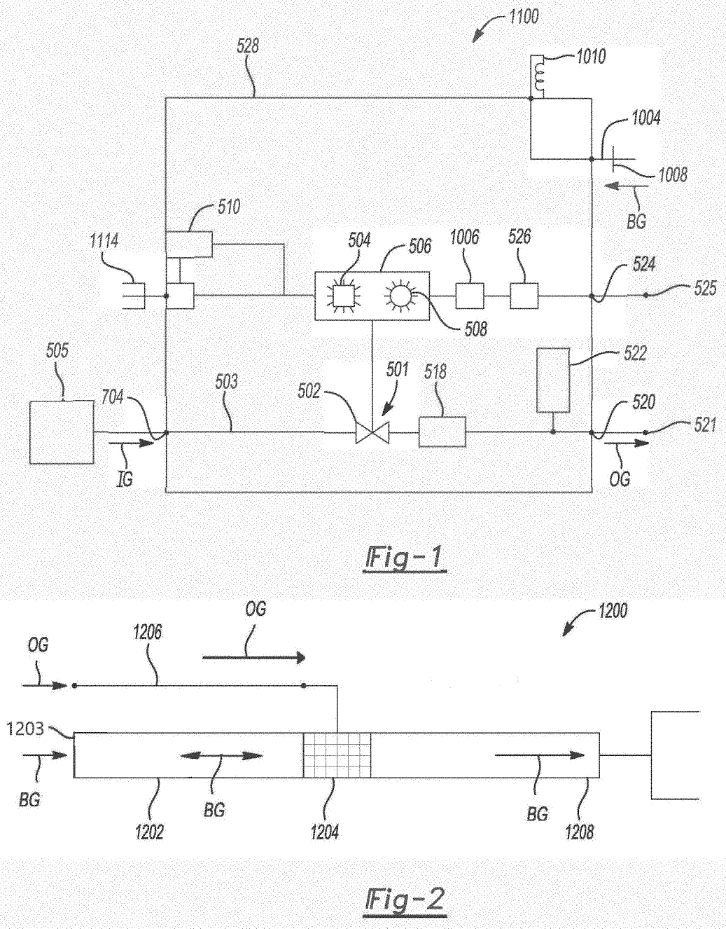

With reference to FIG. 1, a breathing apparatus 1100 includes valve 502, such as an on-off valve or an electronically controlled valve, configured to modulate a flow through a tubing 503. Accordingly, the valve 502 has at least an open state and a closed state. The valve 502 may be part of a valve arrangement 501. The valve arrangement 501 may therefore include one or more of the valves 502. It is also contemplated that the valve arrangement 501 may include other types of valves. Hence, the breathing apparatus 1100 can include valve 502 to minimize cost and weight. The breathing apparatus 1100 functions by receiving input gas IG from an input gas source 505 through the tubing 503. The tubing 503 is fluid communication with the input gas source 505 to allow the input gas source 505 to supply the input gas IG to the breathing apparatus 1100. As non-limiting examples, the input source 505 may be an air compressor, air blower, stationary oxygen concentrator, portable oxygen concentrator, air tank, and/or oxygen tank. A continuous flow of input gas IG enters the breathing apparatus 1100 through the tubing 503, and when the valve 502 opens, the flow rate of input gas IG and output gas OG is the same or at least substantially the same.

The ON-OFF cycles of the valve 502 are controlled using a controller 504, such as a microprocessor or microcontroller unit. The controller 504 may be part of an electronic board 506, which can contain additional electronic components including but not limited to: power electronics, resistors, capacitors, alarms 508, and copper traces. The electronic board 506 may include one or more alarms 508. The alarms 508 can, for example, be used to warn the user of one or more of the following conditions: tubing disconnections, electrical or air supply failure, high peak airway pressure, auto-positive end-expiratory pressure (auto-PEEP), high gas supply pressures, and/or no spontaneous breathing. Further, this electronic board 506 may be utilized as a battery management system for a portable ventilator device that is battery powered.

The breathing apparatus 1100 can include an electrical power source 510, such as a portable rechargeable Li-Ion battery pack or another suitable portable battery assembly. As a non-limiting example, Nickel Metal Hydride (NiMH) Rechargeable Batteries and an 8-battery holder may comprise the electrical power source 510. This is electrically designed to be a 12V circuit as a battery backup in case of main power supply failure, which makes the power electronics on the electronics board 506 simpler. The electrical power source 510 may be recharged after use by AC power module operation when the main power supply is back online. Each AAA cell is 1.2V with a rated capacity of 800 mAH. These alkaline batteries are safe and effective. A power receptacle 1114 is electrically connected to the electrical power source 510 and can function as a recharging interface, such as a port or cable, thereby allowing the electrical power source 510 to be recharged. As non-limiting examples, the power receptacle 1114 may be a Universal Serial Bus-C (USB-C), a USB, a micro-USB, or other charging interfaces. The electrical power source 510 may be electrically connected to the electric board 506 to supply electricity to the controller 504 and the alarms 508.

This controller 504 can be in the form of an FPGA, MCU, single board computer, ASIC, PLC on a chip, and/or other processing or computer hardware that can control the ON/OFF or OPEN/CLOSE cycles of a solenoid valve 502. The valve 502 can be controlled using fluidic chips or other non-conventional or pneumatic methods of valve control, such as air cylinder actuations. For example, an air cylinder or pressure actuator and a check valve can replace the valve 502.

The pressured output gas OG may be outputted in a plurality of different waveforms, such as descending ramp, ascending ramp, sinusoidal, and/or square wave form, among others. Further, these output gas waveforms and flow rates may be adjusted based on breathing airway pressure and/or flow measurements from a second lumen air line. In the presently disclosed breathing apparatus 1100, the flow control and breathing measurements are separately obtained via dual lumen airlines. This dual lumen airline setup prevents electrical signal interference and saturation of the gas output pressure/flow and the breathing measurement pressure/flow sensor sensors found in prior art oxygen conserving devices and ventilators. Further, this also allows for the use of much more sensitive pressure sensors for detecting breathing. In other mechanical ventilators, single lumen tubes are used and, as such, the flow output and breath “triggering” or detection are done in the same airline. Further, in other breathing apparatus, only inhalation is detected. In other breathing apparatus, exhalation and inhalation breathing flows are spearhead using one-way check valves which comprise the dual limb ventilator circuit. In the mechanical breathing apparatus 1100 of the present disclosure, the proximal pressure line is bidirectional (i.e., there are no check valves) and, as such, there is no pressure or flow “triggers” but rather patterns in breathing are mathematically computed based on nasopharynx pressure and/or breath detection sensor waveforms. In experimental use, by positioning the pressure sensors for breath detection in a separate lumen from the lumen used for gas output, it was found six times (6x) more sensitive pressure sensors can be utilized with a dual lumen setup for detecting breathing compared to single lumen pressure sensors. The breathing apparatus 1100 may also have rest, exercise, and/or sleep settings.

The flow rate of this continuous output gas OG to the patient is measured using a flow sensor 518. This flow sensor 518 is in fluid communication with the tubing and can include a plurality of sensor methodologies. For example, the flow sensor 518 may utilize the thermo-transfer principle, also known as the calorimetric principle, to measure large ranges of gas flow rates when the gain factor of the flow sensor 518 is specifically calibrated and tested, such that the sensor output is amplified and two point trimmed at zero flow as well as a secondary flow rate point to optimize linearity within a certain flow rate range, such as 0-40 standard liter per minute (SLPM) gas flow. Under this thermo-transfer principle, inside the flow sensor module 518, a temperature sensor (not shown) is heated periodically by a heater element (not shown). The flowing gas absorbs heat energy and conducts it away. The resulting temperature change is an indication of flow, which translates to an analog voltage value that is then correlated to a flow output curve based on experimental data from the original equipment manufacturer (OEM) or sensor manufacturer during calibration and/or testing. Generally, this flow sensor 518 is a flow-through type sensor, wherein the flow sensor 518 includes a barb fitting inlet that connects to the tubing 503, as well as a barb outlet to the flow outlet airline 520 with minimal resistance of fluidic loss. This flow outlet airline 520 can connect to a 22 mm breathing tube, hose barb, adapter, or other tubing connection thereafter. Further, this flow outlet airline 520 can also be fluidly coupled to an air entrainment device 522. In the present disclosure, the term “air entertainment device” means a physical object configured to entrain a fluid, such as a nozzle, a Venturi conduit, a conduit using the Coanda effect, a conduit using the Jet principle, or another conduit capable of entraining a fluid. The air entrainment device 522 is in direct fluid communication with the tubing 503. The flow sensor 518 is upstream of the air entrainment device 522, and downstream of the valve 502 to allow the flow sensor 518 to provide the controller 504 with reliability sensing data without interference from the air entrainment device 522. Therefore, the controller 504 is in electronic communication with the flow sensor 518 and is programmed to receive data from the flow sensor 518. The controller 504 is in electronic communication with the valve 502 and is programmed to control the valve 502 based on the data received from the flow sensor 518. The flow sensor 518 can alternatively be other types of sensors, such as: turbine-type flow meters, rotometers, and membrane based differential pressure and temperature sensors that can be used to calculate flow rates, which can work especially well for laminar type or large volume/low pressure flows. the flow outlet airline 520 includes an airline outlet 521.

The breathing apparatus 1100 can also include an alarm 508 in the electronic board 506. The alarm 508 can be an auditable alarm designed for medical applications and can be recognized under the International Electrotechnical Commission (IEC) 60601-1-8 standard. This alarm 508 is a component of the electronics board 506 that can include a specially designed speaker-housing assembly with no circuitry. Other alarm types can also be utilized including but not limited to: piezoelectric type speakers, audio amplifiers, and/or electromagnetic speakers. With this alarm 508, the original equipment manufacturer (OEM) only needs to input a simple square wave signal with one frequency component, and the other needed harmonic sound frequencies are generated acoustically. This greatly simplifies implementation of an audible alarm sound in an IEC 60601-1-8 standard since the harmonic peaks are designed to be acoustically equal to the sound level required under IEC 60601-1-8. This alarm 508 relies on the 2nd option for compliance, a melody table listed in Annex F of the IEC 60601-1-8 standard where specific medical conditions/applications are assigned individual melodies. These melodies are essentially little tunes that change in pitch per the tables in Annex F. The objective is that the medical personnel using medical equipment with alarms that use these melodies will become familiar with them which can help the medical personnel respond more quickly and more appropriately when a specific melody alarm sounds. This breathing apparatus 1100 utilizes the alarm 508 to generate high, medium, or low priority warning sounds depending on the condition of the patient or malfunctions with ventilator equipment such as tubing disconnects. The audible sound has a fundamental frequency <1000 Hz, with at least 4 harmonic frequencies within ±15 dB of the fundamental frequency. This alarm 508 has specific waveform and timing requirements for the three priority sounds, which includes a sound rise time specified by the alarm manufacturer. Alarm settings can include, but are not limited to, the following: if O₂ input from inlet 704 flows, but no breathing/exhalation is detected within 6 seconds, sound alarm—low priority; if the electrical power source 510 is being used—medium priority; if O₂ connected in wrong conduit (e.g., breath detection airline 524, flow outlet airline 520, or an CO₂ exhalation conduit 1004), sound alarm—high priority; if the pressure measured during inspiration using peak airway sensor 1006 is less than 40 cmH₂O for more than 3 breaths in a row, sound alarm—high priority; if the CO₂ exhalation conduit 1004 gets disconnected from ventilator 1000 within 6 seconds of assist or control breath output, sound alarm—medium priority; if the flow outlet airline 520 gets disconnected from ventilator 1000 within 6 seconds of assist or control breath, sound alarm—high priority.

During operation, user spontaneous breathing is detected using a separated breath detection airline 524 and an ultra-sensitive pressure sensor 526 for measuring breathing pressures (e.g., nasopharynx pressure). The breath detection airline 524 includes an airline inlet 525. The airline inlet 525 is separated from the airline outlet 521 of the flow outline airline 520 to minimize interference and therefore increase the accuracy of the pressure sensor 526. The pressure sensor 526 is in fluid communication with the breath detection airline 524. This breath detection airline 524 is configured to be connected to a 22 mm breathing tube, hose barb, adapter, or other tubing connections. The breath detection airline 524 is not in fluid communication with the flow outlet airline 520. By fluidly separating the breath detection airline 524 from the flow outlet airline 520, breathing pressures (e.g., nasopharynx pressures) can be measured without signal interference from the pressure/flow output from the breathing apparatus 1100, which would otherwise saturate the ultra-sensitive pressure sensor 526 required to measure the breathing pressures (e.g., nasopharynx pressures) from the user of the breathing apparatus 1100. In other ventilators and oxygen concentrators, a single airline is generally utilized in which a flow or pressure trigger threshold, ex. −0.13 cm H₂O pressure, is used to determine the start of inhalation. This generally creates substantial lag in the ventilator gas output or false breathing triggers. Further, this necessitates the use of far less sensitive pressure sensors to prevent the pressure sensor from getting saturated from the output flow gas from the ventilator. Also, if flow is triggered based on a flow ramp, there can still exist substantial signal interference using a single airline.

In the presently disclosed breathing apparatus 1100, a breath detection software is used to predict transitions in breathing states and breathing time states, for example: transition from inhale to exhale, 70% inhalation time, transition from exhale to inhale, predicted PEEP based on % of exhalation. This breath detection software functions by measuring breathing pressures (e.g., nasopharynx pressures) using a separated breath detection airline 524, then storing the voltage values from the pressure sensor 526 in the controller 504 (e.g., microcontroller) RAM or EEPROM. For this reason, the controller 504 is in electronic communication with the pressure sensor 526. Breath transition states and timing predictions are detected through one or more mathematical calculations involving the pressure sensor voltage data including but not limited to: data filtering, differentiation, integration, linear regression analysis and linearizations, moving average calculations, Taylor series approximations, steady state error compensation, model predictive control, proportional control, fuzzy control theory, ODEs, radial basis functions, quadratic-program approximation, feedforward control, adaptive control, PI and/or PID control, SISO control schema, and Laplace transformations. A moving average calculation can be used such that, if the filtered pressure sensor data falls below the moving average, a transition from an inhale to an exhale is predicted.

Other sensors can also be used independently, in combination with, or to replace the pressure sensor(s) 526 described herein to measure data trends in breathing, implement predictive breath detection software algorithms, and/or actuate at certain threshold values and/or ramps including but not limited to: flow sensors, CO2 gas concentration sensors, O2 gas concentration sensors, temperature sensors, humidity sensors, volume sensors, and/or acoustic sensors. This breath detection is used to determine when to output the output gas OG, which can include compressed air, oxygen, or a mixture thereof, to the patient at the correct time in order to provide pressure/ventilatory support, as well as facilitate effective lung gas exchange, ventilation, and manage arterial blood gases (ABGs) such as PaCO₂ and PaO₂. Accordingly, the pressure sensor 526 is configured to generate sensor data indicative of breathing by the user, and the controller 504 is programmed to detect the breathing of the user based on the sensor data received from the pressure sensor 526.

The components and electromechanical subassemblies of the breathing apparatus 1100 are contained within an electronics enclosure 528, which can be manufactured using a plurality of manufacturing methods including but not limited to: injection molding, 3D printing, CNC machining, sheet metal fabrication, PCBA, wire harnessing, and other manual or automated manufacturing techniques not described herein.

With continued reference to FIG. 1, the breathing apparatus 1100 includes an electrical power source 510 (e.g., battery) inside the enclosure 528. The electrical power source 510 is electrically connected to the controller 504. The breathing apparatus 1100 further includes a power receptacle 1114 electrically connected to the electric power source 510, the controller 504, and the electric board 506. The breathing apparatus 1100 does not include CO₂ exhalation valve. For invasive ventilation, a single limb circuit would be required. This type of configuration would be more suited for breathing apparatus with a focus on non-invasive home ventilation, where the capability of optional but less frequent use invasive ventilation is desired. This configuration without the active CO₂ exhalation valve inside the breathing apparatus 1100 substantially reduces power consumption and weight compared to the other breathing apparatus, allowing for lightweight portability with battery power. The breathing apparatus 1100 includes an CO₂ exhalation conduit 1004 configured to receive exhalation gas BG from the user. The inlet 704, the flow outlet airline 520, the breath detection airline 524, and the CO₂ exhalation conduit 1004 can include tubing connectors. For example, the inlet 704, the flow outlet airline 520, the breath detection airline 524, and the CO₂ exhalation conduit 1004 can include quick change connectors such that modifications to the patient circuit and/or gas source can be made, allowing components to be replaced. The CO₂ exhalation conduit 1004 is configured to receive exhalation gases from the user. The breathing apparatus 1100 includes the air entrainment device 522, which in some configurations is a fixed FiO₂ based on mechanical design and hence should be easy to remove and replace in order for a user to adjust FiO₂. The breathing apparatus 1100 includes a bacteria/viral filter 1008 attached to the CO₂ exhalation conduit 1004. Patient expired gas flows back through bacteria/viral filter 1008, which includes a 22 mm breathing tube connector to minimize exhalation resistance, before coming into contact with any internal device components. This viral/bacterial filter 1008 can include a standard coaxial International Standards Organization (ISO) connectors (e.g., ISO 5356-1 standard) that connect to standard breathing tubes using 15 mm internal diameter (ID)/22 mm outer diameter (OD) connectors for applications in breathing circuits, scavenging circuits, mechanical ventilation, and manual ventilation, including bag valve mask (BVM). This viral/bacterial filter 1008 is designed for single-patient use and, in some embodiments, can have bidirectional airline, be in-line, low flow resistance of 1.5 cm H₂O pressure at 60 LPM, hydrophobic and electrostatic filtering properties, dead space of 45 mL, and ultrasonically welded. An heat and moisture exchange (HME) filter or active heated humidification system and/or airline can be added to the flow outlet airline 520 to heat and moisturize the output gas OG output to the patient in order to prevent drying of airways and promote patient health/comfort. Patient gas is expelled to the atmosphere after flowing through bacteria/viral filter 1008 and through an exhaust muffler 1010. The exhaust muffler 1010 is in communication with the CO₂ exhalation conduit 1004 and is disposed outside the enclosure 528 to safely expel the CO₂ gases.

The breathing apparatus 1100 can include a peak airway pressure sensor 1006 in direct fluid communication with the pressure sensor 526. An LCD screen can indicate, using a graphic or light emitting diode (LED) bar, when adjustments to gas source input flow should be made based on the peak airway pressure sensor measurements measured by the peak airway pressure sensor 1006. Generally, gas source flow input should be increased when SpO₂ saturation is less than 90%, which can be measured using a separate patient/vital signs monitor and/or pulse oximeter and decreased when peak airway pressure is high (i.e., more than 35 cm H₂O). A fixed tidal volume delivered per breath can be provided to user via the LCD screen or via a separate instruction manual based on adjustment of wall O₂ supply flow rates. The user can increase tidal volumes delivered to patient by increasing O₂ flow rate input at the inlet 704. The inlet 704 can be an input gas source connector and can include a barb fitting, diameter-index safety system (DISS) connectors, quick connectors, and others. For example, the input gas source connector can be a ¼″ National Pipe Tapered (NPT) barb fitting that connects to 50 psi hospital wall pipeline O₂ supply or O₂ tank using ¼″ internal diameter (ID) oxygen tubing. The inlet 704, the flow outlet airline 520, the breath detection airline 524, and a CO₂ exhalation conduit 1004 can include tubing connectors. For example, the inlet 704, the flow outlet airline 520, breath detection airline 524, and the CO₂ exhalation conduit 1004 can include quick change connectors such that modifications to the patient circuit and/or gas source can be made, allowing components to be replaced. The CO₂ exhalation conduit 1004 is in direct fluid communication with the viral/bacterial filter 1008 and exhaust muffler 1010 to facilitate filtering and exhausting the exhalation gases outside the enclosure 528. Further, the CO₂ exhalation conduit 1004 is configured to receive exhalation gases from the user of the breathing apparatus 1100. The breathing apparatus 1100 includes the air entrainment device 522, which in some configurations is a fixed FiO₂ based on mechanical design and hence should be easy to remove and replace in order for a user to adjust FiO₂.

With reference to FIG. 2, a non-invasive circuit 1200 can be connected to the breathing apparatus 1100 or other breathing apparatus described herein. The non-invasive circuit includes a breathing tubing 1202 (e.g., 22 mm tubing), an adapter 1204, an oxygen tubing 1206, and a patient interface 1208. This breathing tubing 1202 and any other tubing described herein can have various connector and inner tubing diameter sizes not specified in this disclosure. An inlet 1203 of the breathing tubing 1202 connects to the breath detection airline 524 to minimize flow resistance and measure breathing pressures (e.g., nasopharynx pressures) accurately without signal interference from the oxygen flow. The oxygen tubing 1206 can be connected at the outlet 521 of the airline flow outlet airline 520. The tidal volume from the breathing apparatus 1100 can be output to the patient in a unidirectional flow from the inlet 1203 of the oxygen tubing 1206 to the barb inlet of the adapter 1204, and then to the patient interface 1208 either during a control or assist breath. The adapter 1204 serves as a connection point for the oxygen tubing 1206 and the breathing tubing 1202, allowing tidal volume flow output to the patient interface 1208 as well as bidirectional breath detection software data measurements using the 22 mm breathing tubing 1202 as a flow conduit to the sensors inside the breathing apparatus 1100, such as the pressure sensor 526 with a pressure measurement range of ±0.018 PSIG. The non-invasive circuit 1200 is configured to be disposed outside the enclosure 528.

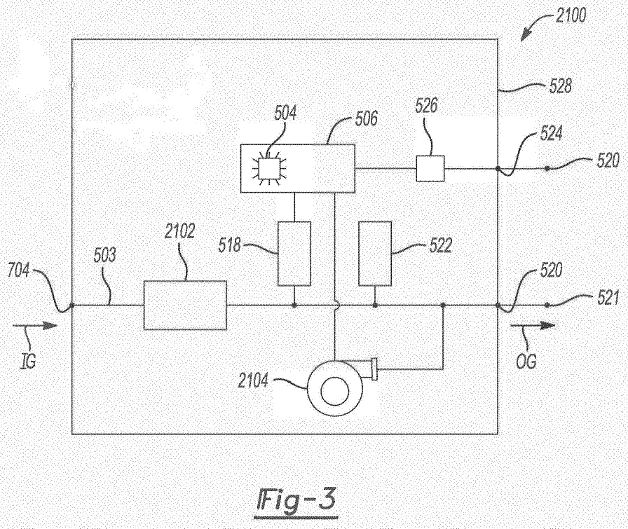

FIG. 3 illustrates a breathing apparatus 2100 that that can function as bilevel positive airway pressure (BiPAP) device or continuous positive airway pressure (CPAP) device, oxygen (O₂) concentrator, and/or breathing apparatus 2100 with different modes. The breathing apparatus 2100 includes an enclosure 528, a tubing 503 configured to receive the input gas IG, and an internal oxygen concentrator 2102 in fluid communication with the tubing 503. The tubing 503 is entirely or at least partially disposed inside the enclosure 528 to minimize the space occupied by the breathing apparatus 2100. The internal oxygen concentrator 2102 is integrated with the breathing apparatus 2100 and is therefore entirely disposed inside the enclosure 528 to minimize the space occupied by the breathing apparatus 2100. The internal oxygen concentrator 2102 can be used to generate enriched oxygen flow to the patient (i.e., output gas OG). The internal oxygen concentrator 2102 can be turned ON or OFF either automatically using electronic control from the controller 504 (e.g., a microcontroller unit) or via user adjustment of a human-computer interface, including, but not limited to, knobs, touchscreens, and/or switches. The internal oxygen concentrator 2102 can produce and/or deliver oxygen on demand based on a patient's breathing needs, provide a continuous flow of oxygen, and/or produce an oscillatory or irregular oxygen output pattern to the user via the flow outlet airline 520.

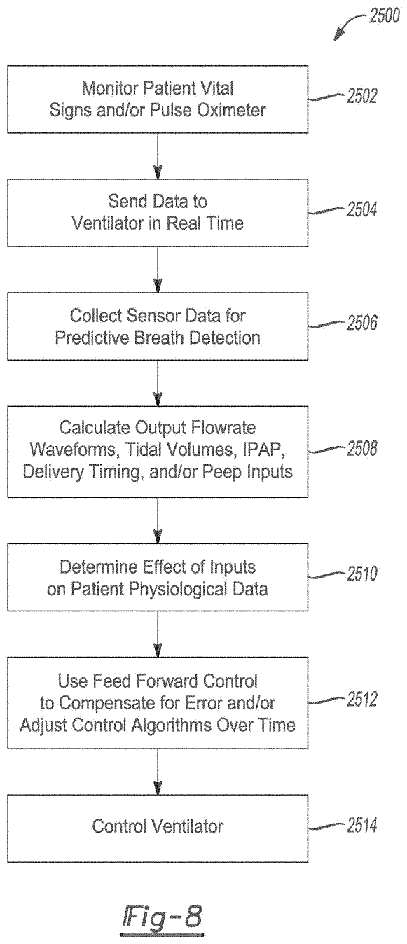

The breathing apparatus 2100 can include an air entrainment device 522 in fluid communication with the internal oxygen concentrator 2102. The air entrainment device 522 is downstream of the internal oxygen concentrator 2102 to entrain the flow of oxygen originating from the internal oxygen concentrator 2102. The enriched oxygen exiting from the oxygen concentrator 2102 can be used to entrain room air using the air entrainment device 522. The breathing apparatus 2100 can additionally include an air blower 2104 in fluid communication with the internal oxygen concentrator 2102 and the tubing 503. The air blower 2104 may be in communication with the controller 504. The controller 504 can be programmed to adjust the output gas OG to the patient by the air blower 2104. The air entrainment device 522 could be substituted for or used in combination with the air entrainment device 522 to perform air-O₂ mixing. In some embodiments, oxygen could be delivered to the patient during useful phases of respiration as measured using the breath detection airline 524 and the pressure sensor 526. After oxygen is delivered during the useful phase of respiration, a positive-end expiratory pressure (PEEP) can be provided using the air blower 2104 to prevent lung collapse in patients with chronic lung diseases, especially those who are mechanically ventilated. This output pressure from the air blower 2104 may be controlled using the controller 504 or via user input from a human-computer interface 2406 (FIG. 7), at specific ranges for example 0.1-20 cmH₂O pressure. The output flow (e.g., output gas OG) may also be controlled using the controller 504. For example, the controller 504 may control the output gas OG by controlling the blower motor speed of the air blower 2104, voltage, and/or power consumption of the breathing apparatus 2100. In some embodiments, an additional pressure sensor can be added to the outlet airline 520 to measure the output pressure of the output gas OG to the patient.

In some embodiments, the pressure of the output gas OG provided to the patient may be controlled by the controller 504 or the user. The air blower 2104 may control the output airflow (e.g., output gas) to modulate the pressure based on a setpoint. For example, if the output pressure of the O₂ and/or compressed air tidal volume from the outlet airline 520 is 6.8 cmH₂O at a flow of 40 LPM and the setpoint is 3.9 cmH₂O, the air blower 2104 can output 1 cmH₂O pressure at 40 LPM flow to achieve the setpoint. In some embodiments of the invention, oxygen pulses could be output intermittently at a frequency greater than an inhalation frequency. In some embodiments, during a period of useful respiration one or more pulse(s) of oxygen could be output followed in terms of timing by one or more pulse(s) of air from the air blower 2104. The lengths of these oxygen and/or blower air pulses can be different or the same as each other.

In another embodiment, the air blower 2104 may be used as an integrated or separate BiPAP/CPAP machine, wherein modes and settings could be selectable, deactivated, and/or activated by the user, healthcare provider, and/or DME based on payment/billing code. For example, the DME supplier may remotely, using software only, enable the breathing apparatus 2100 for use as a non-invasive ventilator if the patient were only prescribed a non-invasive ventilator. If a patient, however, requires supplemental oxygen one year later, the DME can remotely enable this feature using software and then subsequently bill Medicare or an insurance provider for that add-on. In some embodiments, this can also include integrated oxygen and CPAP for obstructive sleep apnea patients with overlap syndrome.

In some embodiments, the blower pressure of the air blower 2104, including IPAP and PEEP, can be controlled, via the controller 504, by the user, clinician, and/or healthcare provider, with the settings recommended or based on the patient prescription and/or real time physiological characteristics such as breathing, pulse oximetry data, vital signs data, etc. For BiPAP, this generally means that the pressures of the air output can range between 5-20 cmH₂O IPAP, and at least 3 cmH₂O less for PEEP, for example 2-17cmH₂O PEEP. These IPAP and PEEP variables can be independently or jointly controlled, by the machine software itself, clinician, and/or user. For CPAP or IPAP, the pressure for IPAP and PEEP would be the same. Hence, only one pressure setpoint would be set. In one embodiment, tidal volume and flow rates of the air blower 2104 could also be controlled by the controller 504 (e.g., microprocessor) of the breathing apparatus 2100, a clinician, and/or the user to maximize user comfort, with guidelines based on the patient interface used which could vary from user to user based on patient physiology and mask leakage. This PEEP could also be determined based on peak airway pressure or predicted using the breath detection software. In some embodiments, the breathing apparatus 2100 can also include wireless communication technology and/or features that allow the breathing apparatus 2100 to function as an at-home sleep test, and/or at-home oxygen test, and provide patient monitoring for the clinician.

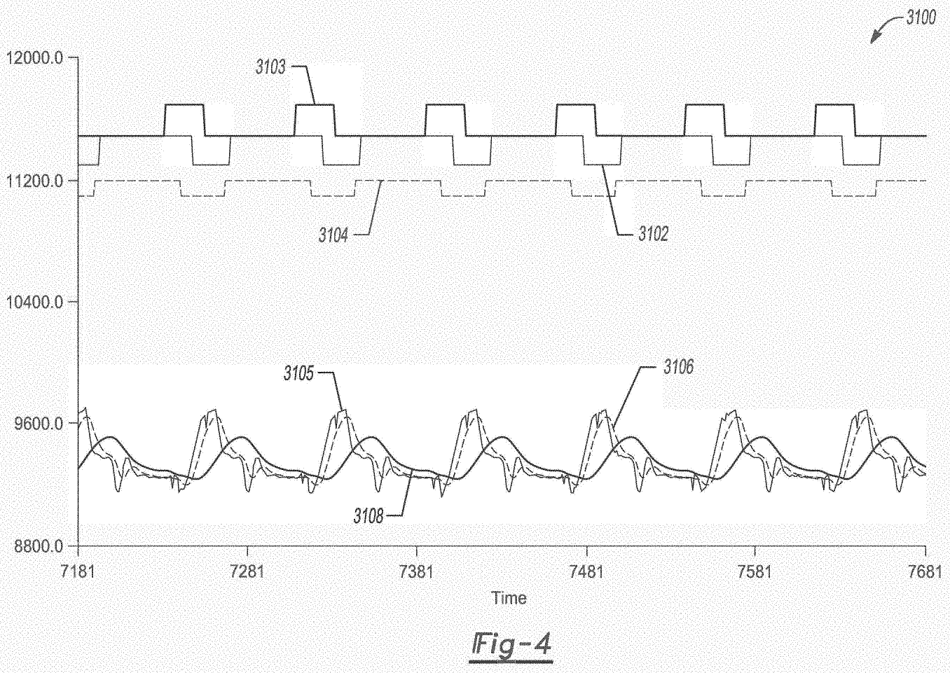

FIG. 4 illustrates a breath detection predictive curve software graphs 3100. In FIG. 4, the horizontal axis represents time. Further, in FIG. 4, the first breath detection graph 3102 at the top represents software that predicted breath using measurements from the pressure sensor 526. As discussed above, the pressure sensor 526 can measure breathing pressures (e.g., nasopharynx pressure) from the user of a breathing apparatus, such as breathing apparatus 1100. However, it is envisioned that the breath detection predictive software graphs 3100 along with the method (or algorithm) 2500 can be executed in breathing apparatus 1100 or other breathing apparatus described herein. As discussed above, the breathing apparatus 1100 (or other breathing apparatus described herein) can be, but not limited to, Continuous Positive Airway Pressure (CPAP) machine or Bilevel Positive Airway Pressure (BiPAP) machines. Thus, the presently disclosed predictive breath detection software can be used with any breathing apparatus capable of supplying output gas OG to a user. The second detection graph 3104 at the top represents lung simulator breath detection using an IngMar Medical ASL5000™ lung simulator. The graph 3103 at the top represents the flow output to the user during testing determined based on the first detection graph 3102, the second detection graph 3104, or both. The controller 504 is programmed with a control algorithm for predicting breathing and outputting flow at certain specified times. This control algorithm may include a lag compensation algorithm. This lag algorithm can allow for the correction of systemic inaccuracies in predictive breath detection, such as overshoot, and allow for flow output to be output, for example, earlier than predicted by the breath detection software. Thus, the controller 504 is programmed to generate a flow output graph (e.g., graph 3103) as a function of time based on the first detection graph 3102 and/or the second breath detection graph 3104 and command the breath apparatus 1100 (or any other breathing apparatus described herein) to supply output gas OG to the user in accordance with the flow output graph 3103. The actual pressure data graph 3105 at the bottom represents actual sensor data (obtained from the pressure sensor 526) in terms of voltage readings. The actual sensor data obtained from the pressure sensor 526 can be filtered (with for example a low pass filter) and is represented by the filtered pressure sensor data graph 3106. Using the filtered pressure sensor data, a predictive curve graph 3108 can be generated. In some embodiments, the controller 504 can calculate a moving average based on past breaths detected. If the moving average is less than the filtered pressure sensor data 3106, then an inhalation is predicted. If the moving average is equal to or greater than the filtered pressure sensor data 3106, then an exhalation is predicted. The controller 504 can then command the breathing apparatus, such as, but not limited to, the mechanical breathing apparatus 1100, to supply the output gas OG to the user on a breath by breath basis based on the predicted exhalation and inhalation pattern of the user.

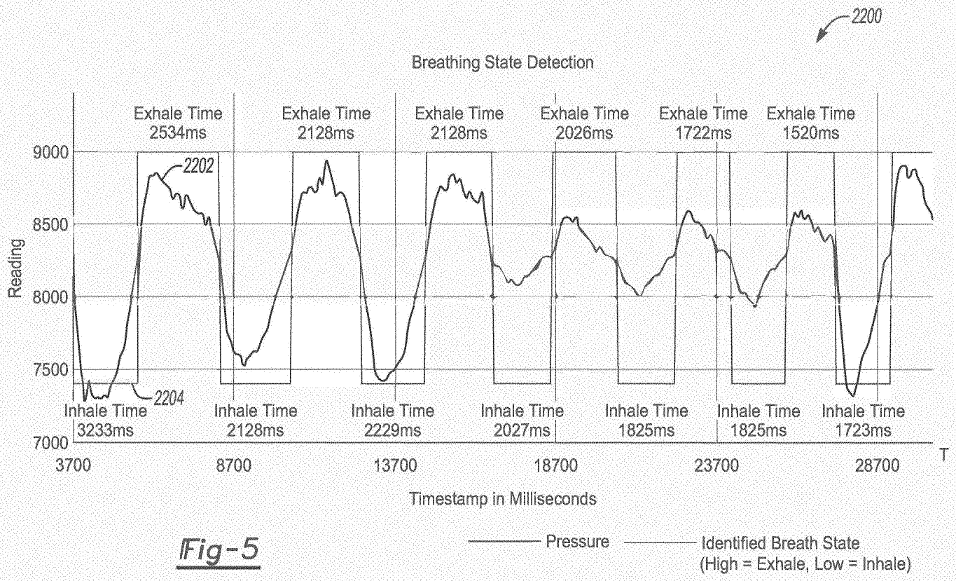

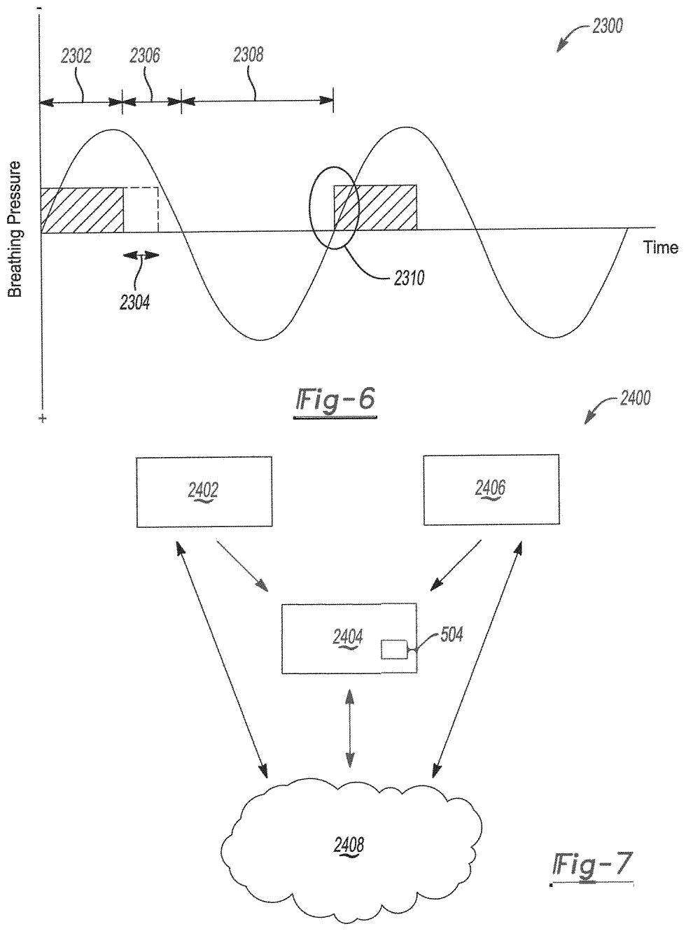

FIG. 5 illustrates that breathing timing can be predicted using measurements from the pressure sensor 526 as shown in the graph 2200. The graph 2200 plots the measurements or readings 2202 of the pressure sensor 526 versus time T. For example, during the first breath 2202, an inhalation time of 3233 milliseconds is predicted and an exhalation time of 2534 milliseconds is predicted. The readings 2202 of the pressure sensor 526 at each time T may be saved to the controller 504 and/or may be uploaded to a cloud-based software 2408 (FIG. 6). In the graph 2200, the horizontal axis represents time stamps. The readings 2202 can be used by a control algorithm for pattern analysis or treatment. As shown by the plot 2204, the control algorithm may determine that inhalation occurs when the reading 2202 is equal to or less than a predetermined minimum threshold, and exhalation occurs when the reading 2202 is equal to or greater than a predetermined maximum threshold. The readings 2202 are time stamped and, the controller 504 and/or the cloud-based software 2408 may determine an inhalation and exhalation pattern based on the time stamped reading 2202. As a non-limiting example, the readings 2202 may be time stamped continuously or every 5000 milliseconds to generate a breath pattern that will be used to predict the inhalation of the user. However, it is envisioned that the readings 2202 may be time stamped at different time intervals. Additionally, or alternatively, the readings 2202 may be time stamped at specific transition points. For example, the readings 2202 may be time stamped when the reading 2202 is equal to or less than the predetermined minimum threshold (and inhalation is detected), and when the readings 2202 are equal to or greater than the predetermined maximum thresholds (when exhalation is detected). However, it is contemplated that the readings 2202 can be time stamped in real time (i.e., continuously) to detect the inhalation and exhalation by the user as well as the duration of the inhalation and exhalation. The readings 2202 may be used for traceability or correlation with other body measurements. In a non-cloud configuration, the control algorithm and the data collected are not stored on the cloud-based software system 2408 but rather on the controller 504 of the breathing apparatus 2404 and/or the controllers of the patient or vital sign monitor 2402 and/or the pulse oximeter 2406, or in a database in communication with one or more of these, and/or any other suitable breathing apparatus. In other words, the data and control algorithm can be localized into the controller (e.g., controller 504) or a local server of a breathing apparatus 1100.

FIG. 6 illustrates a tidal volume graph 2300 compared with breathing cycle and ventilator device operations. This tidal volume graph 2300 is meant to represent that of a breathing apparatus 1100 (or another breathing apparatus described herein). As illustrated in FIG. 6, a tidal volume is output during a period of the user's respiration (time period 2302 while the valve 502 is open). In spontaneous breathing patients, this period of respiration is generally near the end of exhalation (for example the 90% exhalation time to approximately the 70% time of inhalation), because the last 30% of the inhalation (time period 2302) is anatomical deadspace where no useful gas exchange in the lungs occurs. Further, this tidal volume output can be a fixed and/or variable volume (for example between 50-2000 mL of gas) and can be adjusted by the user manually or may be adjusted automatically by the breathing apparatus, such as, but not limited to, the breathing apparatus 1100. In some embodiments, the variable volume output can be adjusted on a breath by breath basis by the controller 504 based on user physiological data such as heart rate, blood pressure, PaO₂, PaCO₂, and/or breathing gas concentrations from the user which can include oxygen %, nitrogen %, CO₂%, and/or trace gas concentrations. Some of this physiological data can be collected using a patient monitor and/or pulse oximetry system 2402, 2406 (FIG. 7). This physiological data can be stored on the controller 504 and/or the cloud-based software 2408. The physiological data may be correlated with the breathing timing data collected to optimize, for example, the flow rate of the output gas OG supplied to the user of the breathing apparatus 1100 (or another breathing apparatus described herein). In some embodiments, the patient monitor and/or pulse oximetry system may interface with the breathing apparatus 1100 (or another breathing apparatus described herein) via wired or wireless communication. This tidal volume may be a fixed and/or variable time duration. In some embodiments, the variable time duration of tidal volume output may be during spontaneous breathing (assist breath). Alternatively, the tidal volume output may be fixed tidal volume output during non-spontaneous breathing (control breath). The duration of tidal volume output may be time controlled, pressure controlled, flow controlled, volume controlled, and/or a combination of these control methodologies. When this tidal volume is being output to the patient during the time period 2302, the valve 502 opens. After the tidal volume is output to the patient, the valve 502 closes during a time period 2306, such that an inspiratory hold time is created. This inspiratory hold time is generally 30% of tidal volume output duration timing (time period 2304) and is meant to facilitate gas exchange in the lungs, as well as measure plateau and peak airway pressures to evaluate the breathing apparatus pressure support provided to the patient. This monitoring of plateau and peak airway pressures can allow the user to adjust tidal volumes and/or inspiratory flows to minimize the risk of barotrauma or associated lung injuries (VALI). After the inspiratory hold time is complete, the CO₂ exhalation conduit 1004 opens for the duration of exhalation (i.e., time period 2308) until a new tidal volume is output by the breathing apparatus 1100 (or another breathing apparatus described herein). In one embodiment, PEEP can be predicted based on the exhalation time of the tidal volume output, which is based on the measurement or estimation of expiratory pressures as well as pressure of tidal volume gas. This tidal volume output start time for predictive breath detection, for example 98% exhalation time as shown in area 2310, can be adjusted breath by breath by the ventilator 1000 such that breathing synchrony for the patient improves over time during use.

With reference to FIG. 7, a cloud-based breath detection software and patient monitoring system 2400 is illustrated. By way of example, a “cloud-based” system, as that term is used herein, can refer to a system which includes software and/or data which is stored, managed, and/or processed on a network of remote servers hosted in the “cloud”, e.g., via the Internet, rather than on local severs or personal computers. The system 2400 includes a patient or vital sign monitor 2402, a breathing apparatus 2404 (e.g., a ventilator, CPAP, a BiPAP or another device subtle to provide output gas OG to a user) in communication with the patient or vital sign monitor 2402, and a pulse oximeter 2406 in communication with the breathing apparatus 2404. The pulse oximeter 2406 is configured to monitor and communicate oxygen saturation or oxygen levels in the blood of a patient. The patient or vital sign monitor 2402 is configured to monitor and communicate patient data, such as vital signs including but not limited to blood pressure, SpO₂, and heart rate to the breathing apparatus 2404 (e.g., breathing apparatus 1100). The breathing apparatus 2404 is in communication with cloud-based software 2408 and has internal electronics that allow for input data connections such as a serial bus, Internet of Things (IoT), and/or wireless communications capabilities. The breathing apparatus 2404 can be electronically designed such that one or more I/O ports can be used at the same time, for example allowing for simultaneous receipt of data from the patient monitor 2402 and the pulse oximeter 2406. In some embodiments, the patient monitor 2402 can send this monitored data to the breathing apparatus 2404 using wireless communication for example Bluetooth or Wi-Fi, or wired communication such as a Serial bus, LAN, USB, and/or other means of wired communication to send data in real time, with a small amount of lag such as 50 ms, and/or send previous data stored in patient monitor RAM or EEPROM for analysis by the breathing apparatus 2404. This lag time and sampling rate for data sent to the breathing apparatus 2404 can be fixed or variable, along with amount/time duration of data stored, can be determined based on the processing capabilities of the patient monitor 2402. A secure digital (SD) card or other non-volatile memory format may be used to store a patient profile of vital sign and/or breathing data from the breathing apparatus 2404, which can be analyzed separately either locally on a computer and/or using cloud software 2408 for use by a healthcare provider. The predictive breath detection software can be a cloud based software program 2408. The analysis and computations related to the pressure sensor 526 and other breathing sensor data is done in the cloud-based software 2408 using one or more control algorithms. The controller 504 inside the breathing apparatus 2404 receives a set of instructions from the cloud software 2408, such as but not limited to “OPEN Valve 1 at 00:03:07 time”, “allow flow of 6 LPM of oxygen for 1.375 seconds”, etc. The cloud-based software 2408 can communicate with the breathing apparatus 2404 using a variety of internet or communication protocols such as Wi-Fi, LAN, Wi-LAN, Bluetooth, 3G, 4G LTE, 5G, VPN, wireless hotspots, and/or other means. The breathing apparatus 2404 may have an integrated IoT device to allow low latency wireless communication with the cloud software 2408. The predictive breath detection software may be executed in the cloud-based software system 2408 and/or the internal controller 504 of the breathing apparatus 2404. As such, the cloud-based software system 2408 may only be used to store data, such as the data collected as described above with respect to FIGS. 26 and 27. In other words, the data collected may reside on the cloud-based software system 2408. To this end, the cloud-based software system 2408 may be in communication with the controller 504 of the breathing apparatus 2404, a patient or vital sign monitor 2402 and/or a pulse oximeter 2046. This collected data may include, among other things, readings 2202 as well as the time stamps shown in FIG. 4. The data stored on the cloud-based software system 2408 may then be communicated to the controller 504 of the breathing apparatus 2404. In this case, the control algorithm resides in the controller 504 of the breathing apparatus 1100. Therefore, the controller 504 of the breathing apparatus 2404 executes the control algorithm based on the data received from the cloud-based software system 2408. Alternatively, the cloud-based software 2408 not only stores the data collected as described above with respect to FIGS. 4 and 5, but also executes the analysis and computations required to predict breathing by the user and calculates, among other things, the flow rate of the output gas OG supplied to the user based on that analysis and computations. In other words, in this case, the cloud-based system 2408 stores the data collected and executes the control algorithm. In this case, the cloud-based software system 2408 communicates the flow rate of the output gas OG to the controller 504 of the breathing apparatus 2404. The controller 504 then commands the breathing apparatus 2404 to output the output gas OG at the flow rate determined by the cloud-based software system 2408. It is also envisioned that the data may reside in different devices, such as the controllers of a patient or vital sign monitor 2402 and/or the pulse oximeter 2406 and then communicated to the controller 504 of the breathing apparatus 2404. The controller 504 of the breathing apparatus 2404 may also store data relating to the pressure measurements of the pressure sensor 526.

The controller 504 and/or the cloud-based software system 2408 can include and/or be in communication with a memory and a database for receiving, storing and/or providing data from the pressure sensors 526, the pulse oximeter 2406, and/or the patient monitor 2402. In addition, the controller 504 and/or the cloud-based software system 2408 may include a central processing unit (not shown) for executing the method 2500. The memory of the controller 504 and/or the cloud-based software system 2408 may at least partially be tangible and non-transitory (e.g., ROM, RAM, EEPROM, etc.) and may be of a size and speed sufficient, for example, to execute the method 2500, storing the database, and/or communication with the breathing apparatus 2404, the controller 504, the pressure sensor 526, the pulse oximeter 2406, and/or the patient monitor 2406. The examples provided herein are non-limiting. For example, it would be understood that the functions of the cloud-based software system 2408 may be provided by a single server, or may be distributed among multiple servers, including third party servers, and that the data within the cloud-based software system 2408 may be provided by databases configured other than as described for the database. For example, the event duration data and/or process parameters related to breathing apparatus 2404 may reside in a shared database stored in the controller cloud-based system 2408 in communication with the server 20. The database may be distributed among multiple servers, including third party servers, in communication with each other and the server 20 through a network (not shown), such as the Internet, and/or directly.

FIG. 8 is a flowchart of a method 2500 for using the breathing apparatus 2404 that dynamically adjusts tidal volumes based on PaO₂ saturations and vital sign monitor data. The method 2500 begins at block 2502. At block 2502, the vital signals of the patient are monitored with the patient or vital sign monitor 2402. The patient or vital sign monitor 2402 then generates sensor sign data indicative of the monitored vital signs of the patient. Also at block 2502, the oxygen saturation or oxygen level in the blood of the patient is monitored using the pulse oximeter 2406. The pulse oximeter 2406 then generates sensor oxygen data indicative of the monitored oxygen saturation or oxygen level in the oxygen of the patient. It is envisioned that the vital signs and the oxygen saturation or oxygen level in the blood may be measured using integrated or separate devices such as vital sign monitors, patient monitors, pulse oximeters, EtCO₂ sensors, external or internal flow, pressure, and/or gas concentration sensors.

Then, the method 2500 proceeds to block 2504. At block 2504, the sensor sign data generated by the patient or vital sign monitor 2402 and the sensor oxygen data generated by the pulse oximeter 2406 is communicated to the controller 504 of the breathing apparatus 2404 in real time. The controller 504 of the breathing apparatus 2404 therefore receives the sensor sign data generated by the patient or vital sign monitor 2402 and the sensor oxygen data generated by the pulse oximeter 2406 in real time. Then, the method 2500 continues to block 2506.

At block 2506, the pressure sensor 526 measures and monitors the breathing pressures (e.g., nasopharynx pressure) of the patient. The pressure sensor 526 generate breathing sensor data indicative of the breathing pressure of the patient. This breathing sensor data is then communicated to the controller 504 of the breathing apparatus 2404. The controller 504 then collects the breathing sensor data generated by the pressure sensor 526 in real time. After collecting breathing sensor data, the method 2500 continues to block 2508.

At block 2508, the controller 504 of the breathing apparatus 2404 and/or the cloud-based software system 2408 uses the breathing sensor data to, among other things, predict breathing by the patient as discussed above with respect to FIG. 4. In addition, the controller 504 of the breathing apparatus 2404 calculates and/or predicts O₂ flow rate waveforms, tidal volumes, IPAP, delivery timing, inhalation/exhalation (I:E) ratios, and/or PEEP inputs based on a variety of data measurements including but not limited to: predictive breath detection using pressure sensor measurements, breathing flow rate measurements, vital signs from a vital sign and/or patient monitor 2402 and/or pulse oximeter 2406, such as heart rate, blood pressure, and/or SpO₂, pulse oximetry data regarding arterial blood gases such as SpO₂, SpCO₂, PaO₂, EtCO₂, and/or PaCO₂, breathing gas concentration percentage of elements or compounds such as O₂ percentage, N₂ percentage, CO₂ percentage, and/or trace gases in ppm. After block 2508, the method 2500 proceeds to block 2510.

At block 2510, the controller 504 of the breathing apparatus 2404 and/or the cloud-based software system 2408 determines the effect of changing inputs, such as O₂ flow rates, on patient physiological data, such as SpO₂, PaCO₂, heart rate, blood pressure. After block 2510, the method 2500 proceeds to block 2512.

At block 2512, the controller 504 of the breathing apparatus 2404 and/or the cloud-based software system 2408 then uses feedforward control to compensate for error and/or adjust control algorithms over time. As a result, physicians or healthcare providers do not need to manually adjust ventilator controls, such as PEEP, IPAP, etc. The method 2500 then proceeds to block 2514.

At block 2514, the controller 504 commands the breathing apparatus 2404 to supply the output gas OG to the user at predetermined times based on the predicted breathing by the user. In other words, the controller 504 controls the breathing apparatus 2404 on a breath by breath basis based on the times for the next predicted inhalation. For instance, the controller 504 may command the valve 502 to open a certain amount of time (e.g., 2 milliseconds) before the next predicted inhalation of the user is about to occur. As a result, the control of the breathing apparatus 2404 takes into account the other steps of the method 2500, such as the feed forward compensation for errors and/or adjusts the output gas to be supplied to the user. Thus, the controller 504 is programmed to control the output gas OG supplied to the user based on the data collected, among other things, from the pressure sensor 526, the pulse oximeter 2406, and/or the patient or vital sign monitor 2402. This control of the output gas OG may include commanding the breathing apparatus 2404 to supply the output gas OG at a certain flow rate as calculated in block 2508 and corrected in block 2512.

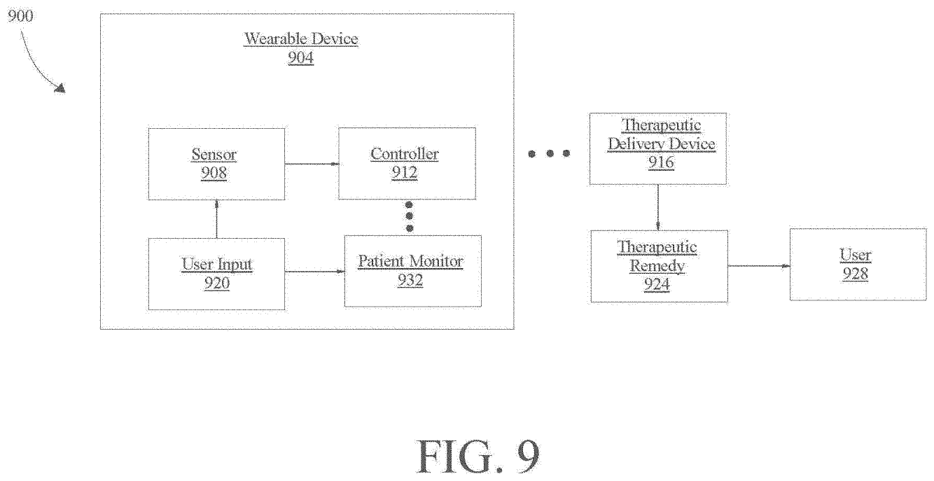

Referring now to FIG. 9, a cloud-based user physiology detection software and patient monitoring system 900 is shown. System 900 may include wearable device 904. Cloud-based user physiology detection software and patient monitoring system 900 may include a component of breathing apparatus 1100 as described above in FIGS. 1-8. Wearable device 904 may be worn by user 928. In some embodiments, wearable device 904 may include a plurality of fastening devices. A plurality of fastening devices may include, but is not limited to, straps, zippers, Velcro, magnetic holders, belts, notches, adhesives, and the like. In some embodiments, wearable device 904 may include, but is not limited to, a hat, a watch, a ring, glasses, a face mask, a face covering, eyewear, or other wearable items. In some embodiments, wearable device 904 may include a power source. A power source may include one or more battery cells. In some embodiments, wearable device 904 may include a solar cell. In other embodiments, wearable device 904 may include a power source that may be configured to receive power wirelessly. In other embodiments, wearable device 904 may include a power source that may be configured to receive power through a wired connection. In some embodiments, wearable device 904 may be configured to connect to an external power supply. An external power supply may include an external battery, power outlet, and the like.

In some embodiments, and with continued reference to FIG. 9, wearable device 904 includes sensor 908. Sensor 908 may include sensors in the form of individual sensors or a plurality of sensors working as a unit or individually. Examples of sensor 908 may include a heart rate sensor, temperature sensor, humidity sensor, conductivity sensor, respiratory rate sensor, gas pressure sensor, blood gas sensor, blood pressure sensor, and/or accelerometer, or a combination thereof. Sensor 908 may be configured to receive user input 920. In some embodiments, user input 920 may include physiological data. As used in this disclosure, “physiological data” are a form of one or more measurements of a user relating to one or more biological functions, anatomical systems, and/or mental states, or the like. In some embodiments, physiological data may relate to a physiological state of a user. A “physiological state” as used in this disclosure is any condition of a biology, mentality, and the like of a user. A physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. In some embodiments, physiological data may include, but is not limited to, a skin temperature, a blood pressure, a heart rate, a breathing rate, a blood gas level, an acceleration, and the like. In some embodiments, wearable device 904 may be configured to receive physiological data from an external computing device in the form of a self-reported questionnaire, a survey, a data input from a doctor, a survey from a doctor, and the like.

Still referring to FIG. 9, wearable device 904 may include controller 912. Controller 912 may include any computing device as described in this disclosure, including without limitation a microcontroller, microprocessor, digital signal processor (DSP) and/or system on a chip (SoC) as described in this disclosure. Controller 912 may include, be included in, and/or communicate with a mobile device such as a mobile telephone or smartphone. Controller 912 may include a single computing device operating independently, or may include two or more computing device operating in concert, in parallel, sequentially or the like; two or more computing devices may be included together in a single computing device or in two or more computing devices. Controller 912 may interface or communicate with one or more additional devices as described below in further detail via a network interface device. Network interface device may be utilized for connecting controller 912 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Controller 912 may include but is not limited to, for example, a computing device or cluster of computing devices in a first location and a second computing device or cluster of computing devices in a second location. Controller 912 may include one or more computing devices dedicated to data storage, security, distribution of traffic for load balancing, and the like. Controller 912 may distribute one or more computing tasks as described below across a plurality of computing devices of computing device, which may operate in parallel, in series, redundantly, or in any other manner used for distribution of tasks or memory between computing devices. Controller 912 may be implemented using a “shared nothing” architecture in which data is cached at the worker, in an embodiment, this may enable scalability of system 900 and/or controller 912.

With continued reference to FIG. 9, controller 912 may be designed and/or configured to perform any method, method step, or sequence of method steps in any embodiment described in this disclosure, in any order and with any degree of repetition. For instance, controller 912 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Controller 912 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

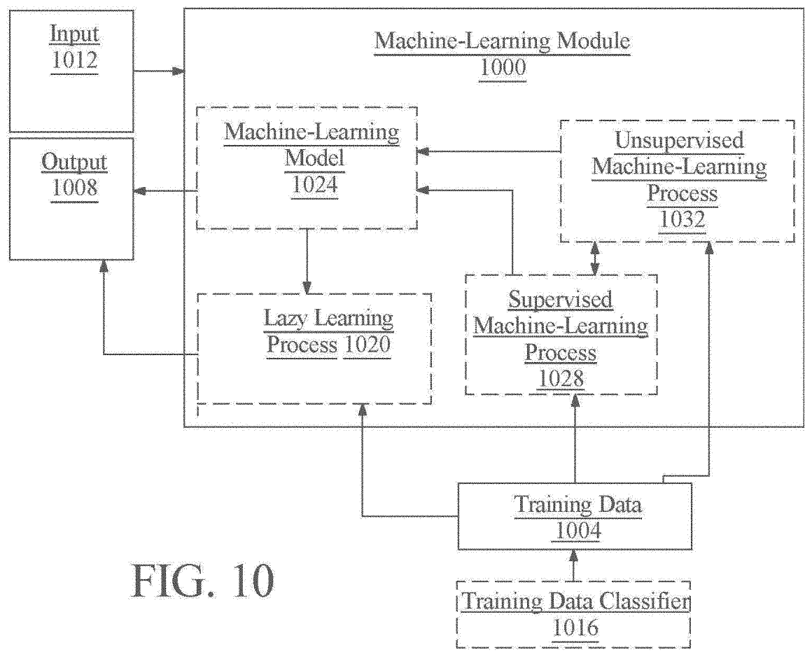

Still referring to FIG. 9, controller 912 may be configured to receive data from sensor 908. Controller 912 may be configured to communicate data received by one or more external computing devices. In some embodiments, controller 912 may communicate data to a cloud-based network. A cloud-based software may be configured in a cloud-based network may be configured to interpret data from sensor 908. A cloud-based network may be as described above in FIGS. 1-8. In some embodiments, controller 912 may be in electronic communication with therapeutic delivery device 916. In some embodiments, controller 912 may communicate with therapeutic delivery device 916 through a cloud-based network. A “therapeutic delivery device” as used in this disclosure is any device configured to administer a therapeutic remedy to a user. Therapeutic delivery device 916 may include a computing device. In some embodiments, therapeutic delivery device 916 may include a housing for medications. In some embodiments, therapeutic delivery device 916 may be configured to include an output component. An “output component” as used in this disclosure is any device configured to receive an input from therapeutic delivery device and administer a remedy. An output component may be configured to administer a therapeutic remedy 924. A “therapeutic remedy” as used in this disclosure is any form of treatment for a user. In some embodiments, an output component may include, but is not limited to, a dispensing device, a syringe, a needle, an air tube, or the like. In some embodiments, therapeutic delivery device 916 may include a breathing device. A breathing device may include, but is not limited to, a ventilator, continuous positive air pressure machine, inhaler, or other breathing device. In some embodiments, controller 912 may be configured to communicate a command to therapeutic delivery device 916. The command may include instructions to administer therapeutic remedy 924 to user 928. Controller 912 may be configured to modify therapeutic remedy 924 as a function of data received from sensor 908. In some embodiments, controller 912 may modify therapeutic remedy 924 as a function of a therapeutic delivery classifier. A therapeutic delivery classifier may be configured to classify a therapeutic delivery for a user. A classifier may be as described in detail below with reference to FIG. 10. In some embodiments, a therapeutic delivery classifier may be configured to correlate physiological data of user 928 with an appropriate therapeutic remedy 924. In a non-limiting example, a therapeutic delivery classifier may correlate lower blood oxygen saturation levels of user 928 with a therapeutic remedy 924 that may include an oxygen delivery system. Classifying a therapeutic delivery for a user may include utilizing a machine learning process. A machine learning process may be as described in further detail below with reference to FIG. 10.

Still referring to FIG. 9, sensor 908 may be configured to determine a heart rhythm of user 928. Controller 912 may send a command to therapeutic delivery device 916 to administer therapeutic remedy 924 based on a heart rhythm of user 928. In a non-limiting example, controller 912 may send a command to therapeutic delivery device 916 to administer therapeutic remedy 924 during an atrial contraction of a user 928. In some embodiments, therapeutic remedy 924 may include a heart rhythm medication, such as, but not limited to, calcium channel blockers, and/or beta channel blockers. In some embodiments, sensor 908 may be configured to determine a breathing pattern of user 928. A “breathing pattern” as used in this disclosure is any repeating metric of a respiratory function of a user. Controller 912 may send a command to administer therapeutic remedy 924 based on a breathing pattern of user 928. Therapeutic delivery device 916 may include an oxygen delivery system. Sensor 908 may detect an inspiration and/or expiration of user 928. Sensor 908 may detect an optimal output gas delivery time. An “optimal output gas delivery time” as used in this disclosure is any period of a delivery of an output gas that is timed with a breathing pattern of a user. Controller 912 may send a command to therapeutic delivery device 916 to administer therapeutic remedy 924 during an inhalation of user 928 which may include, but not limited to oxygen delivery, air pressure delivery, medication delivery, and the like. In some embodiments, sensor 908 may detect a sleep pattern of a user. A “sleep pattern” as used in this disclosure is any metric of a repeating oscillation between random eye movement (REM) and non-random eye movement (REM) stages of sleep. A sleep pattern may include, but is not limited to, stages of sleep, duration of sleep, start of sleep, end of sleep, and the like. In some embodiments, sensor 908 may determine a circadian rhythm of user 928. A “circadian rhythm” as used in this disclosure is any change in an individual over a 24 hour cycle. A circadian rhythm may include physical, mental, and/or behavioral changes that follow a 24 hour cycle. Controller 912 may be programmed to modify therapeutic remedy 924 as a function of a detected sleep pattern of user 928. In a non-limiting example, sensor 908 may determine user 928 is in a stage of random eye movement (REM) sleep. In a nonlimiting example, controller 912 may communicate with therapeutic delivery device 916, such as, but not limited to a continuous positive air pressure (CPAP) system to administer therapeutic remedy 924 in the form of a pressure adjustment of the gas delivered to user 928. In another non-limiting example, sensor 908 may determine user 928 is in a non-REM sleep stage. Controller 912 may signal therapeutic delivery device 916 to administer therapeutic remedy 924 in the form of awaking user 928, such as by alarms, lights, and/or vibration devices. In some embodiments, controller 912 may communicate with therapeutic delivery device 916 where therapeutic delivery device generates a recommended sleep schedule for user 928. Therapeutic delivery device 916 may generate a recommended sleep schedule and transmit the recommended sleep schedule to one or more GUIs. In some embodiments, a display may include, but is not limited to, a smartphone display, computer monitor, laptop, tablet, and the like. In some embodiments, therapeutic delivery device 916 may transmit a recommended sleep schedule to a cloud-based network. In some embodiments, controller 912 may be programmed to detect a detrimental effect threshold. A detrimental effect threshold, as used herein, is a measurement of physiological data of user 928 that indicates when the measurement of physiological data may fall outside of a predetermined range of values. For instance and without limitation, a detrimental effect may correspond to a negative physiological impact. A negative physical impact may include, but is not limited to, low blood pressure, low oxygen levels, abnormal breathing rate, irregular heartbeat, and the like. In some embodiments, a detrimental effect threshold may be measured in, but is not limited to, a heart rate, temperature, oxygen levels, medication amount, heart rhythm, breathing rate, breathing pressure, breathing cycle, and the like. In some embodiments, controller 912 may be programmed to alert user 928 that a detrimental effect threshold has been reached. In a non-limiting example, sensor 908 may detect low oxygen levels of user 928, such as 88% blood oxygen saturation. Controller 912 may send an alert to user 928 through wearable device 904. Controller 912 may send an alert to a medical professional through a cloud-based network that user 928 has, for example, low oxygen levels. An alert may include, but is not limited to, a vibration, light, alarm, and the like. In another non-limiting example, sensor 908 may detect an irregular heart rate of user 928. Controller 912 may be programmed to send an alert to user 928 through wearable device 904.

With continued reference to FIG. 9, wearable device 904 may communicate through a cloud-based network to emergency providers, which may include, but are not limited to, doctors, physicians, nurses, EMT staff, and the like. In some embodiments, controller 912 may be programmed to activate therapeutic remedy 924 through therapeutic delivery device 916 as a function of a measured detrimental effect threshold. In a non-limiting example, controller 912 may be programmed to active therapeutic delivery device 916 to administer therapeutic remedy 924 in the form of oxygen delivery if oxygen saturation levels of user 928 drop below 90%. In some embodiments, wearable device 904 includes patient monitor 932. A “patient monitor” as used in this disclosure, is any device configured to track vital signs of a user. Patient monitor 932 may include one or more sensors configured to measure a vital sign of user 928. A vital sign may include, but is not limited to, body temperature, blood pressure, breathing rate, and/or heart rate. Patient monitor 932 may be programmed to continuously monitor one or more vital signs of user 932. In some embodiments, patient monitor 932 may be in electronic communication with controller 912. Patient monitor 932 may communicate data about a vital sign of user 928 to controller 912. Controller 912 may be programmed to send an alert to user 928 and/or emergency services about a vital sign of user 928. In some embodiments, controller 912 may be programmed to activate therapeutic delivery device 916 to deliver therapeutic remedy 924 as a function of data received from patient monitor 932. In some embodiments, patient monitor 932 may be configured to display monitoring data to user 928. “Monitoring data” as used in this disclosure, is any continuously tracked metric of a user. In some embodiments, monitoring data may include vital signs. In some embodiments, patient monitor 932 may be configured to display monitoring data of user 928. In some embodiments, patient monitor 932 may be configured to display monitoring data of user 928 through wearable device 904 and/or through an external display connected to a cloud-based network. A patient monitor and cloud-based network may be as described above in FIGS. 1-8.

Still referring to FIG. 9, in some embodiments, controller 912 may be configured to communicate data to a smartphone, laptop, desktop, monitor, or other device. Data communicated by controller 912 may include, but is not limited to, therapy administered, therapeutic remedies, therapeutic remedy options, physiological data measured, predicted physiological trends and/or patterns, and the like. In some embodiments, user 928 may select therapeutic remedy 924 through a graphical user interface (GUI) of a display in communication with controller 912. In some embodiments, sensor 908 may determine a pattern of physiological data of user 928. A “pattern of physiological data” as used in this disclosure, is any repeating metric measured of a user. A pattern of physiological data may include, but is not limited to, skin temperature, motion, heart rate, breathing rate, oxygen levels, and the like. A pattern of physiological data may correspond to an activity, such as, but not limited to, sleeping, exercising, meditating, and the like. In some embodiments, a pattern of physiological data may include an exercise pattern. An “exercise pattern” as used in this disclosure is any repeating form of engagement in physical activity. In some embodiments, therapeutic remedy 924 may include medication for pain, asthma, diabetes, blood thinners, and the like. In some embodiments, therapeutic delivery device 916 may include an aerosol generator. An aerosol generator may be configured to transform a liquid medication into an aerosol that may be included in therapeutic remedy 924.

Referring now to FIG. 10, an exemplary embodiment of a machine-learning module 1000 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 1004 to generate an algorithm that will be performed by a computing device/module to produce outputs 1008 given data provided as inputs 1012; this is in contrast to a non-machine learning software program where the commands to be executed are determined in advance by a user and written in a programming language.

Still referring to FIG. 10, “training data,” as used herein, is data containing correlations that a machine-learning process may use to model relationships between two or more categories of data elements. Training data 1004 may be received from an external computing device. In some embodiments, training data 1004 may be received from previous inputs and/or outputs of machine learning module 1000. For instance, and without limitation, training data 1004 may include a plurality of data entries, each entry representing a set of data elements that were recorded, received, and/or generated together; data elements may be correlated by shared existence in a given data entry, by proximity in a given data entry, or the like. Multiple data entries in training data 1004 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1004 according to various correlations; correlations may indicate causative and/or predictive links between categories of data elements, which may be modeled as relationships such as mathematical relationships by machine-learning processes as described in further detail below. Training data 1004 may be formatted and/or organized by categories of data elements, for instance by associating data elements with one or more descriptors corresponding to categories of data elements. As a non-limiting example, training data 1004 may include data entered in standardized forms by persons or processes, such that entry of a given data element in a given field in a form may be mapped to one or more descriptors of categories. Elements in training data 1004 may be linked to descriptors of categories by tags, tokens, or other data elements; for instance, and without limitation, training data 1004 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 10, training data 1004 may include one or more elements that are not categorized; that is, training data 1004 may not be formatted or contain descriptors for some elements of data. Machine-learning algorithms and/or other processes may sort training data 1004 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 1004 to be made applicable for two or more distinct machine-learning algorithms as described in further detail below. Training data 1004 used by machine-learning module 1000 may correlate any input data as described in this disclosure to any output data as described in this disclosure. As a non-limiting illustrative example inputs of machine-learning module 100 may include physiological data and outputs may include patterns of physiological data such as breathing patterns. As another non-limiting example, inputs may include physiological data and outputs may include therapeutic remedies.

Further referring to FIG. 10, training data may be filtered, sorted, and/or selected using one or more supervised and/or unsupervised machine-learning processes and/or models as described in further detail below; such models may include without limitation a training data classifier 1016. Training data classifier 1016 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 1000 may generate a classifier using a classification algorithm, defined as a processes whereby a computing device and/or any module and/or component operating thereon derives a classifier from training data 1004. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1016 may classify elements of training data to physiological data, therapeutic remedies, and/or biological functions.

Still referring to FIG. 10, machine-learning module 1000 may be configured to perform a lazy-learning process 1020 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1004. Heuristic may include selecting some number of highest-ranking associations and/or training data 1004 elements. Lazy learning may implement any suitable lazy learning algorithm, including without limitation a K-nearest neighbors algorithm, a lazy naive Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 10, machine-learning processes as described in this disclosure may be used to generate machine-learning models 1024. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 1024 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 1024 may be generated by creating an artificial neural network, such as a convolutional neural network comprising an input layer of nodes, one or more intermediate layers, and an output layer of nodes. Connections between nodes may be created via the process of “training” the network, in which elements from a training data 1004 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 10, machine-learning algorithms may include at least a supervised machine-learning process 1028. At least a supervised machine-learning process 1028, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include physiological data as described above as inputs, therapeutic remedies as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 1004. Persons skilled in the art, upon reviewing the entirety of this disclosure, will be aware of various possible variations of at least a supervised machine-learning process 1028 that may be used to determine relation between inputs and outputs. Supervised machine-learning processes may include classification algorithms as defined above.

Further referring to FIG. 10, machine learning processes may include at least an unsupervised machine-learning processes 1032. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 10, machine-learning module 1000 may be designed and configured to create a machine-learning model 1024 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 10, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

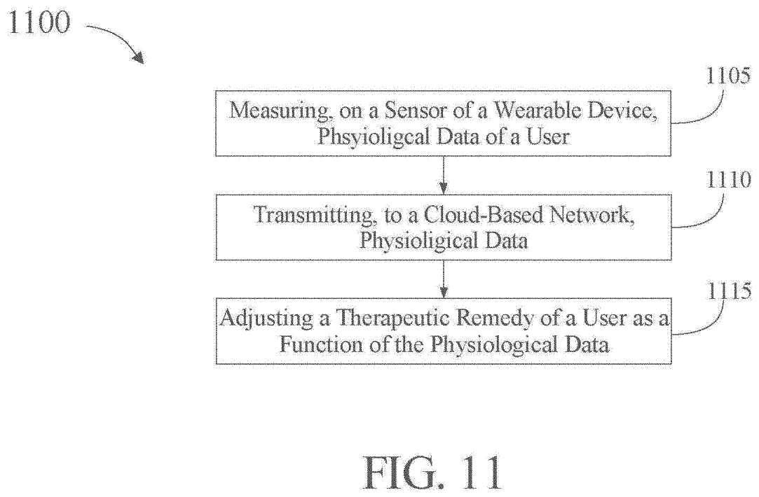

Now referring to FIG. 11, an exemplary embodiment of a method 1100 of monitoring a physiology of a user is disclosed. At step 1105, method 1100 includes measuring on a sensor of a wearable device physiological data of a user. A wearable device may include a mask, ring, watch, glasses, and the like. In some embodiments, a sensor of a wearable device may be configured to measure physiological data of a user. Physiological data may correspond to biological functions of a user. This step may be implemented, without limitation, as described in FIGS. 1-11.

Still referring to FIG. 11, at step 1110, method 1100 includes transmitting to a cloud-based network physiological data. Physiological data may be transmitted from a wearable device. In some embodiments, physiological data may be transmitted to a plurality of devices of a cloud-based network. This step may be implemented, without limitation, as described in FIGS. 1-11.

Still referring to FIG. 11, at step 1115, method 1100 includes adjusting a therapeutic remedy of a user as a function of physiological data. A therapeutic remedy may be administered from a therapeutic delivery device. In some embodiments, a therapeutic remedy may include, but is no limited to, delivery of oxygen, breathing gas, medication delivery, and the like. Breathing gas may include, but is not limited to, pressurized ambient air, non-pressurized ambient air, ambient air mixed with a medicine, ambient air mixed with concentrated oxygen, and the like. This step may be implemented, without limitation, as described in FIGS. 1-11.

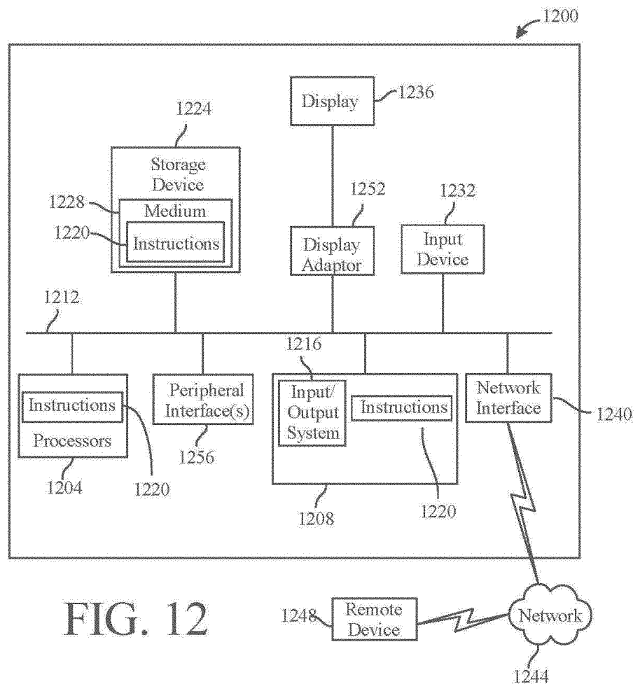

FIG. 12 shows a diagrammatic representation of one embodiment of a computing device in the exemplary form of a computer system 1200 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1200 includes a processor 1204 and a memory 1208 that communicate with each other, and with other components, via a bus 1212. Bus 1212 may include any of several types of bus structures including, but not limited to, a memory bus, a memory controller, a peripheral bus, a local bus, and any combinations thereof, using any of a variety of bus architectures.

Processor 1204 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1204 may be organized according to Von Neumann and/or Harvard architecture as a non-limiting example. Processor 1204 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1208 may include various components (e.g., machine-readable media) including, but not limited to, a random-access memory component, a read only component, and any combinations thereof. In one example, a basic input/output system 1216 (BIOS), including basic routines that help to transfer information between elements within computer system 1200, such as during start-up, may be stored in memory 1208. Memory 1208 may also include (e.g., stored on one or more machine-readable media) instructions (e.g., software) 1220 embodying any one or more of the aspects and/or methodologies of the present disclosure. In another example, memory 1208 may further include any number of program modules including, but not limited to, an operating system, one or more application programs, other program modules, program data, and any combinations thereof.

Computer system 1200 may also include a storage device 1224. Examples of a storage device (e.g., storage device 1224) include, but are not limited to, a hard disk drive, a magnetic disk drive, an optical disc drive in combination with an optical medium, a solid-state memory device, and any combinations thereof. Storage device 1224 may be connected to bus 1212 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1224 (or one or more components thereof) may be removably interfaced with computer system 1200 (e.g., via an external port connector (not shown)). Particularly, storage device 1224 and an associated machine-readable medium 1228 may provide nonvolatile and/or volatile storage of machine-readable instructions, data structures, program modules, and/or other data for computer system 1200. In one example, software 1220 may reside, completely or partially, within machine-readable medium 1228. In another example, software 1220 may reside, completely or partially, within processor 1204.

Computer system 1200 may also include an input device 1232. In one example, a user of computer system 1200 may enter commands and/or other information into computer system 1200 via input device 1232. Examples of an input device 1232 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1232 may be interfaced to bus 1212 via any of a variety of interfaces (not shown) including, but not limited to, a serial interface, a parallel interface, a game port, a USB interface, a FIREWIRE interface, a direct interface to bus 1212, and any combinations thereof. Input device 1232 may include a touch screen interface that may be a part of or separate from display 1236, discussed further below. Input device 1232 may be utilized as a user selection device for selecting one or more graphical representations in a graphical interface as described above.

A user may also input commands and/or other information to computer system 1200 via storage device 1224 (e.g., a removable disk drive, a flash drive, etc.) and/or network interface device 1240. A network interface device, such as network interface device 1240, may be utilized for connecting computer system 1200 to one or more of a variety of networks, such as network 1244, and one or more remote devices 1248 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1244, may employ a wired and/or a wireless mode of communication. In general, any network topology may be used. Information (e.g., data, software 1220, etc.) may be communicated to and/or from computer system 1200 via network interface device 1240.

Computer system 1200 may further include a video display adapter 1252 for communicating a displayable image to a display device, such as display device 1236. Examples of a display device include, but are not limited to, a liquid crystal display (LCD), a cathode ray tube (CRT), a plasma display, a light emitting diode (LED) display, and any combinations thereof. Display adapter 1252 and display device 1236 may be utilized in combination with processor 1204 to provide graphical representations of aspects of the present disclosure. In addition to a display device, computer system 1200 may include one or more other peripheral output devices including, but not limited to, an audio speaker, a printer, and any combinations thereof. Such peripheral output devices may be connected to bus 1212 via a peripheral interface 1256. Examples of a peripheral interface include, but are not limited to, a serial port, a USB connection, a FIREWIRE connection, a parallel connection, and any combinations thereof.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware that enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Accordingly, the disclosure and the figures are to be regarded as illustrative rather than restrictive.

What is claimed is:

1. A cloud-based user physiology detection software and patient monitoring system, comprising:
a wearable device, wherein the wearable device comprises:
a sensor in electrical communication with the wearable device, wherein the sensor is configured to receive physiological data from a user; and
a patient monitor configured to monitor a vital sign of a user of the wearable device;
a therapeutic delivery device configured to administer a therapeutic remedy to the user; and
a computing device in communication with the therapeutic delivery device and the wearable device, wherein the computing device is configured to modify a therapeutic remedy of the therapeutic delivery device as a function of the physiological data.

2. The system of claim 1, further comprising a therapeutic delivery classifier, wherein the therapeutic delivery classifier is configured to:
receive training data, wherein the training data correlating user physiological sample data to a plurality of therapeutic delivery data;
train a machine learning model using the training data; and
classify, as a function of the machine learning model, a therapeutic delivery for a user wherein the machine learning model is configured to input a plurality of user physiological sample data and output a therapeutic delivery for a user.

3. The system of claim 1, wherein the computing device is configured to calculate a detrimental effect threshold.

4. The system of claim 3, wherein the wearable device is configured to alert the user of a measured detrimental effect.

5. The system of claim 1, wherein the wearable device is configured to communicate data to the computing device through a cloud-based network.

6. The system of claim 5, wherein the cloud-based network is configured to store data from the wearable device.

7. The system of claim 1, wherein the computing device is programmed to predict a breathing pattern of a user as a function of physiological data received from the sensor.

8. The system of claim 1, wherein the computing device is configured to display monitoring data to a user.

9. The system of claim 1, wherein the therapeutic delivery device comprises a breathing apparatus, wherein the breathing apparatus comprises:
a tubing configured to receive an input gas;
a flow outlet airline in fluid communication with the tubing, wherein the flow outlet airline is configured to supply an output gas to a user; and
a breath detection airline, wherein the breath detection airline is configured to receive breathing gas from the user via the breath detection airline.

10. The system of claim 1, wherein the therapeutic remedy of the therapeutic delivery device includes a delivery of a breathing gas.

11. The system of claim 1, wherein the therapeutic delivery device comprises a medication delivery system configured to deliver a medication.

12. The system of claim 1, wherein the computing device is programmed to detect an optimal output gas delivery time as a function of a measured breathing cycle of the user.

13. The system of claim 1, wherein the wearable device is configured to track a sleep pattern of the user.

14. The system of claim 13 wherein the wearable device is configured to adjust the therapeutic remedy as a function of the tracked sleep pattern.

15. The system of claim 1, wherein the computing device is programmed to determine a pattern of input from a user based on data of a measured user input.

16. The system of claim 15, wherein the pattern of input includes an exercise pattern.

17. The system of claim 15, wherein a pattern of input includes a circadian rhythm.

18. The system of claim 1, wherein the wearable device includes a ring.

19. The system of claim 1, wherein the wearable device includes a watch.

20. A method of providing a therapeutic remedy using a cloud-based detection software, comprising:
measuring, on a sensor of a wearable device, physiological data of the user;
transmitting, to a cloud-based network, the physiological data of the user; and
adjusting a therapeutic remedy of a user as a function of the physiological data.
